(19) United States
(12) Patent Application Publication
(10) Pub. No.: US 2009/0131772 A1
(43) Pub. Date: May 21, 2009

(54) MEDICAL DEVICE

(75) Inventors: GERHARD SPEKOWIUS, ROETGEN (DE); BERNARDUS HENDRIKUS WILHELMUS HENDRIKS, EINDHOVEN (NL); GERHARDUS WILHELMUS LUCASSEN, EINDHOVEN (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/067,299

(22) PCT Filed: Sep. 18, 2006
(86) PCT No.: PCT/IB2006/053337

§ 371 Date: Mar. 19, 2008

(30) Foreign Application Priority Data
Sep. 20, 2005 (EP) 05300758.9

(51) Int. Cl. A61B 5/1455; A61M 39/00

(52) U.S. Cl. 600/316; 604/8; 600/317

(57) ABSTRACT

A medical device (1) is in the form of a flexible tube (1) having a first end (2) and a second end (3) each adapted for insertion at separate points through the skin (4) of a body into a blood vessel (5), such that the tube (1) defines a conduit that traverses at least in part above the skin (4) of the body. In use, blood flow is diverted from the body through the tube (1) and back to the body. The fluid flowing through the tube (1) above the skin is available for continuous analysis, for example, by a Raman spectroscopy system (10) that analyses the blood for glucose concentration.

The present invention relates to a medical device and in particular, but not exclusively, to a medical device in the form of a tube the ends of which may be inserted into a blood vessel to divert a blood flow temporarily outside of the body, so that the blood can be continuously monitored.

Continuous blood monitoring involves monitoring a patient's blood over an extended time period. It has many important medical applications, for example, the monitoring of glucose levels in the blood of diabetic patients or in other patients who have difficulty in stabilising or controlling blood glucose levels. It may also be used to monitor a hospital patient's blood during and after a period that drugs have been administered to the patient or when the patient is in an intensive care unit. For instance, it may be used for monitoring the effects of antibiotics during sepsis.

There are known non-invasive measuring systems that measure blood glucose concentrations by analyzing the spectra of Raman radiation scattered from a patient's blood vessels. A problem of such systems is that the patient's skin covering the blood vessels scatters too much radiation from the measurement radiation. This results in measurements having a low signal to noise ratio.

Alternative methods involve invasively taking blood samples from a patient at periodic moments in time for analysis. Such discrete sampling is not very convenient for the patients and furthermore can lead to too much blood loss (in particular neonates in incubators).

Embodiments of the present invention aim to alleviate the above-mentioned problems

According to the present invention there is provided a medical device comprising a tube the device having a first end and a second end each adapted for insertion at separate points through the skin of a body, such that when so inserted, the tube defines a conduit that traverses at least in part above the skin of the body, whereby in use, a flow of bodily fluid is diverted from the body through the tube and back to the body, the fluid flowing through the tube above the skin being available for analysis.

Advantageously, this allows for continuous in vivo monitoring of the bodily fluid.

In a preferred embodiment, the first and second ends are adapted for insertion into a blood vessel so that the flow of bodily fluid is a blood flow.

According to the present invention there is also provided medical apparatus comprising: the medical device defined above; and a body fluid monitoring system for monitoring the fluid flowing through the tube above the skin.

In a preferred embodiment the body fluid monitoring system is for monitoring glucose concentrations in blood flowing through the tube above the skin.

An embodiment of the invention will now be described by way of example only with reference to the accompanying drawing in which:

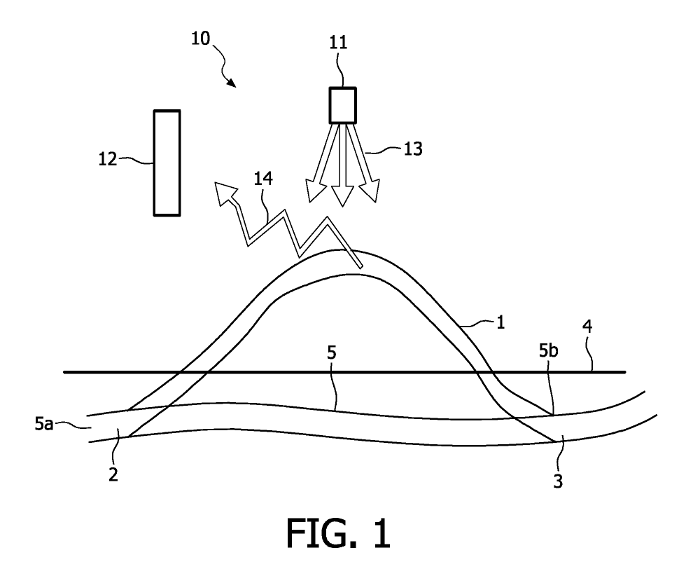

FIG. 1 is a schematic diagram of a system embodying the present invention.

Referring to FIG. 1, there is illustrated a medical device 1 in the form a flexible tube having a first open end 2 and a second open end 3. In use, the first end 2 is insertable through a patient's skin 4 into a blood vessel 5 at a first location 5a. The second end 3 is also insertable through the patient's skin 4 into the blood vessel 5 at a second location 5b down bloodstream of the first location 5a.

In one embodiment the tube is a single integrated device the ends of which can pierce through the skin 4 into the blood vessel 5. Alternatively, a separate instrument may be used to make the incisions through which the ends of the tube are inserted. The tube may also be a modular device, with one or more of the ends of the tube comprising a connector located above the skin during use to which the remainder of the tube is releasably connected. The ends of the tube are placed a few millimetres below the skin.

The tube 1 with its ends 2 and 3 thus inserted into the blood vessel 5 defines a conduit that by passes the blood vessel 5 between the first location 5a and the second location 5b with a portion of the tube 1 extending outside of the skin 4. Blood flows from the blood vessel 5 into the tube 1 through the first end 2 and along the tube 1 outside of the skin 4 before returning to the blood vessel 5 through the second end 3.

The blood flowing through the tube 1 outside of the skin 4 is available for monitoring by monitoring system 10. In a preferred embodiment the monitoring system 10 is a Raman spectroscopy system 10 comprising a radiation source 11 and a detector 12. The radiation source may for example be a lamp, a laser or a light emitting diode and the detector for example a spectrometer. Such Raman monitoring systems are well known to those skilled in the art and will not be discussed in detail herein.

The light source 11 generates a beam 13 that irradiates the blood 15 in part or all of the tube 1 that is outside of the skin 4. This induces a Raman scattering signal 14 that is detected by the detector 12. A measurement of the concentration of a blood constituent of interest, for example glucose, may be obtained from Raman wavelength peaks present in the detected Raman scattering signal 14 associated with that constituent.

Advantageously, as measurements are obtained from above skin level, scattering of the measurement signal by the skin is not an issue and so better signal to noise ratios can be obtained.

Preferably, the material from which the tube is made is transparent to the wavelength range used to monitor for the blood constituent of interest. For example, for Raman scattering monitoring of blood glucose, the material is transparent to radiation of a wavelength range containing one or more Raman peaks associated with glucose. This further improves signal to noise ratio leading to improved accuracy.

Preferably, the material from which the tube is made absorbs radiation from a wavelength spectrum non-relevant to the measurement of the blood constituent of interest. This further improves again signal to noise ratio leading to improved accuracy.

In a preferred embodiment the tube 1 may be coated with a material that helps prevent infections.

The tube 1 may for example be made from a material used to make catheters, for example Teflon (RTM) or the like.

The dimensions of the tube depend upon purpose, but it is envisaged that tube lengths will typically be a few centimetres and tube diameters a few millimetres.

In the above described embodiment Raman spectroscopy is used to monitor the blood. Other techniques may be used, for example, techniques where a radiation source induces Fluorescence from the blood in the tube 1, which Fluorescence is detected and analyzed to obtain a concentration measurement of a blood constituent.

Embodiments of the invention may be used to monitor bodily fluids other than blood.

Embodiments of the invention may be used for continuous blood monitoring of ICU's in hospitals or for other patients requiring monitoring, for example home patients. The invention may also be used in combination with tele-medicin via a home based e-platform.

Having thus described the present invention by reference to a preferred embodiment it is to be well understood that the embodiment in question is exemplary only and that modifications and variations such as will occur to those possessed of appropriate knowledge and skills may be made without departure from the spirit and scope of the invention as set forth in the appended claims and equivalents thereof. In the claims, any reference signs placed in parentheses shall not be construed as limiting the claims. The word “comprising” and “comprises”, and the like, does not exclude the presence of elements or steps other than those listed in any claim or the specification as a whole. The singular reference of an element does not exclude the plural reference of such elements.

1. Medical device comprising:
a tube (1), the tube having a first end (2) and a second end (3) each adapted for insertion at separate points through the skin (4) of a body, such that when so inserted, the tube (1) defines a conduit that traverses at least in part above the skin (4) of the body, whereby in use, a flow of bodily fluid is diverted from the body through the tube (1) and back to the body, the fluid flowing through the tube (1) above the skin (4) being available for analysis.

2. Medical device according to claim 1 wherein the first (2) and second (3) ends are adapted for insertion into a blood vessel (5) so that the flow of bodily fluid is a blood flow.

3. Medical device according to claim 1 wherein the tube (1) is transparent to radiation of a predetermined wavelength range, which in use is used to analyse the fluid flowing through the tube (1) above the skin (4).

4. Medical device according to claim 1 wherein the tube (1) is substantially opaque to radiation having a wavelength outside of a predetermined wavelength range had by radiation which in use is used to analyse the fluid flowing through the tube above the skin (4).

5. Medical apparatus comprising:
the medical device of claim 1; and
a body fluid monitoring system (10) for monitoring the fluid flowing through the tube (1) above the skin (3).

6. Medical apparatus according to claim 5, wherein;
the body fluid monitoring system (10) comprises, a radiation source (11) for illuminating the fluid flowing through the tube (1) above the skin (3); and
a detector (13) for detecting a signal generated by the fluid in response to being illuminated by the radiation.

7. Medical apparatus according to claim 6, wherein;
the body fluid monitoring system (10) is a Raman spectroscopy system.

8. Medical apparatus according to claim 6, wherein;
the detector (12) is for detecting a fluorescence signal generated by the fluid in response to being illuminated by the radiation.

9. Medical apparatus according to claim 5 wherein, the body fluid monitoring system (10) is for monitoring glucose concentrations in blood flowing through the tube above the skin.
